SEQUENCE LISTING (10 sequences)

SEQ ID NO: 1 - protein, 7 aa, synthetic construct
TWLPLPR

SEQ ID NO: 2 - protein, 8 aa, synthetic construct
YVLLPSPK

SEQ ID NO: 3 - protein, 7 aa, synthetic construct
KVPPLLY

SEQ ID NO: 4-7 - skipped (4 entries)

SEQ ID NO: 8 - protein, 7 aa, synthetic construct
TWLPLPR

SEQ ID NO: 9 - protein, 8 aa, synthetic construct
YVLLPSPK

SEQ ID NO: 10 - protein, 7 aa, synthetic construct
KVPPLLY

(19) United States
(12) Patent Application Publication
(10) Pub. No.: US 2025/0213646 A1
(43) Pub. Date: Jul. 3, 2025

(54) BIOACTIVE WALNUT PEPTIDES AND COMPOSITIONS FOR TREATING SKIN

(71) Applicant: L'OREAL, Paris (FR)

(72) Inventors: Rebecca BARRESI, Robbinsville, NJ (US); I-chien LIAO, Princeton, NJ (US); Charbel BOUEZ, Hoboken, NJ (US)

(73) Assignee: L'OREAL, Paris (FR)

(21) Appl. No.: 18/932,713

(22) Filed: Oct. 31, 2024

Related U.S. Application Data
(60) Provisional application No. 63/617,209, filed on Jan. 3, 2024.

(30) Foreign Application Priority Data
Mar. 28, 2024 (FR) 2403133

(51) Int. Cl. A61K 38/08; C07K 7/06

(52) U.S. Cl. CPC A61K 38/08; C07K 7/06

(57) ABSTRACT

Bioactive walnut peptides and use of the bioactive walnut peptides for topical application to the skin are described. The bioactive walnut peptides may be incorporated into a pharmaceutical or cosmetic compositions and are useful for improving skin barrier function.

### CROSS-REFERENCE TO RELATED APPLICATIONS

This application claims benefit of U.S. Ser. No. 63/617,209, filed Jan. 3, 2024, and benefit of French Application No. FR 2403133, filed on Mar. 28, 2024, which are incorporated herein by reference in their entirety.

### REFERENCE TO THE SEQUENCE LISTING

The Sequence Listing submitted Oct. 31, 2024 as an XML file named “Sequence Listing,” created on Oct. 30, 2024 and having a size of 11 kilobytes is hereby incorporated by reference pursuant to 37 C.F.R. § 1.52 (e) (5).

### FIELD OF THE DISCLOSURE

The present disclosure relates to bioactive walnut peptides and use of the walnut peptides to treat skin. The walnut peptides and compositions comprising them are particularly useful for improving skin barrier function, and treating damaged skin, inflamed skin, and skin suffering from damage, disease, or insult.

### BACKGROUND

Skin is a complex organ comprised of multiple cell types and microstructures that work in concert to serve critical functions and support the body's homeostasis. It is the outermost, cornified layer of our body that is primarily responsible for the permeability barrier, protecting against external aggressors and preventing water loss from within. The understanding of the organization, functionality, and underlying mechanisms of the skin barrier has evolved greatly through the years. The formation of an intact and well-maintained stratum corneum, where the permeability barrier resides, relies heavily on the differentiation of epidermal keratinocytes and the synthesis, release, localization, and binding of lipids that include principally ceramides, cholesterol, and free fatty acids. The in-depth research on stratum corneum barrier, its disruption in the pathogenesis of diseases, as well as on barrier responses to environmental insults, has enabled the development of modern therapeutics and topical care routines.

The epidermis maintains its homeostasis and serves critical functions through a dynamic, self-renewing process in which the basal keratinocytes divide and migrate through the stratum spinosum and granulosum while progressively differentiating. When the keratinocytes reach the top of the granular layer, the process of terminal differentiation occurs in which the keratinocytes undergo programmed cell death and flatten out to form the stratum corneum. During this process, the lamellar bodies of granular layer keratinocytes merge with the plasma membranes and release their predominantly lipid contents into the intercellular spaces of the nascent horny layer. An interplay of hydrolytic enzymes and their inhibitors, also excreted via the lamellar bodies, participate in elaboration of the intercellular layered lipid structure and, ultimately, are involved in cell desquamation at the top of the skin. Simultaneous to the extracellular lipid build-up, important changes occur within the keratinocytes upon the formation of stratum corneum. Transglutaminase-1-mediated cross-linking of cytoplasmic proteins at the cell periphery results in the formation of highly insoluble cornified envelopes of the stratum corneum cells, thereafter, called corneocytes. It is followed by a covalent binding to these structures of a monolayer of ceramides, replacing phospholipid plasma membranes of the living cells. These newly formed cornified lipid envelopes constitute the scaffold for further stacking and organization of the intercellular lipids. The composite structure of the stratum corneum, made of corneocytes intercalated by polar lipids, can be compared to a brick-and-mortar wall constituting stratum corneum permeability barrier.

To perform its function as a permeability barrier, the epidermis must remain mechanically resistant while sufficiently flexible to accommodate skin movements and the treadmill-like flow of keratinocytes through the successive layers. Cell-cell and cell-substrate junctions play central roles in the maintenance of mechanical properties of the epidermis. Desmosomes, which interconnect individual cell cytoskeletons into a superstructure, evolve throughout the epithelial tissue, and change their location, protein composition, and glycan distribution according to the stage of the cell differentiation and the occurrence of mechanical constraints. In this process, actin cytoskeleton-bound adherens junctions participate in the dynamics of desmosome and tight junction expression. Upon the stratum corneum formation, these junctions become cross-linked to cornified envelopes and contribute to the enhanced physical resistance of the functional stratum corneum barrier. Mechanical properties of the stratum corneum show a significant increase in stiffness between the deep and superficial corneocytes. The mechanical integrity of the stratum corneum also depends on the direction of the applied shearing forces since lateral, side to side adhesion between the cells is stronger compared to that between the successive corneocyte layers.

The relative impermeability of the stratum corneum and, thus, its barrier function, rely essentially on the intercellular lipids, even though they account for only 15% of the stratum corneum weight. Quasi equimolar proportions of ceramides, cholesterol, and free fatty acids appear to be prerequisite for the correct auto-assembly of the intercellular lipid multilayers within the stratum corneum. The composition of the lipids of the stratum corneum further subdivides into free fatty acids (10%), cholesterol (27%), cholesterol esters (10%), cholesterol sulfate (3%), and ceramides (50%). These lipids organized in multiple bi-layers parallel to the corneocyte surfaces may assemble within the layers into domains presenting different densities. A dense orthorhombic lateral packing of lipid molecules and a more fluid hexagonal format predominate in normal human skin. Efficient filling of stratum corneum interstices is essential for preventing excessive water loss and penetration of environmental contaminants/aggressors.

Formation and restoration of abolished stratum corneum barrier is a dynamic, finely regulated process prone to the influences from intrinsic and environmental factors. In addition to disease conditions and severe environmental exposures from ultraviolet rays or pollution, events that occur in everyday life can also negatively impact the skin barrier. The importance of the stratum corneum in maintaining skin homeostasis, coupled with the prevalence and severity of internal and external factors that can alter its permeability, highlight the need for topical products to support the skin barrier. Understanding of the epidermal permeability barrier structure, composition, and function provides sound foundations for knowledge-based elaboration of topical treatments aimed at the maintenance and improvement of patients' skin in health and disease. Formulating pharmaceutical and cosmetic compositions to improve barrier integrity has been pursued as knowledge surrounding the skin barrier is continuously developing. There is an ongoing effort to address the need for improved barrier function and barrier regeneration.

### SUMMARY OF THE DISCLOSURE

The instant disclosure relates to bioactive walnut peptides and use of the walnut peptides for topical application to the skin. The bioactive walnut peptides, which may be incorporated into a pharmaceutical or cosmetic compositions, improve skin barrier function and reepithelization of skin, repair damaged skin, and improve wound healing. For example, the walnut peptides and compositions comprising them are useful for improving skin barrier function of healthy skin and useful for treating skin that has suffered physical damage, chemical damage, environmental damage, sun damage, and damage caused by disease, including inflammation.

Walnuts are one of the most widely distributed and oldest nuts in the world. They have high nutritional value and are rich in oleic acid, linoleic acid, α-linolenic acid, and other unsaturated fatty acids, vitamins, and proteins. Walnuts are commonly used to make walnut oil because they contain a high lipid content. The residue remaining after lipid extraction is considered a by-product although it contains walnut protein and other useful components. Walnut protein is mainly composed of albumin, globulin, gliadin and glutenin.

Bioactive walnut peptides can be derived from walnut proteins or can be synthesized. For example, walnut peptides are obtained via enzymatic hydrolysis, fermentation hydrolysis, or chemical hydrolysis of walnut proteins or synthetically produced, for example, by solid-phase synthesis. Bioactive peptides that are particularly useful according to the instant disclosure typically have a molecular weight less than 6,000 Da and often less than 1,000 Da. Such walnut peptides can have from 2 to 50 amino acid residues but typically have from 2 or 3 amino acid residues up to about 20 amino acid residues.

Preferably, the bioactive walnut peptides have a minimum of 2 or 3 amino acid residues up to about 20 amino acid residues and impart a positive physiological or dermatological effect on skin cells. Bioactive peptides include amino acids joined by covalent bonds, also referred to as amide or peptide bonds, whereas proteins are polypeptides with a greater molecular weight (MW), i.e., having more than 50 amino acid residues. Bioactive walnut peptides usually display hormone or drug-like activities and are classified based on their mode of action. Many bioactive peptides share some structural features for example, an amino acid residue length for 2 to 20 amino acids.

Useful bioactive walnut peptides according to the instant disclosure include often include one or more amino acid residues selected from leucine, proline, or combinations thereof. In further embodiments, the walnut peptides include three or more amino acid residues selected from leucine, proline, or combinations thereof. Nonlimiting examples of amino acid residues within the walnut peptides that include leucine and proline include Leu-Pro-Leu (LPP), Leu-Leu-Pro, Pro-Pro-Leu (PPL), and Pro-Leu-Pro (PLP). Further nonlimiting examples of amino acid sequence that can be included in the bioactive walnut peptides include Thr-Trp-Leu-Pro-Leu-Pro-Arg (TWLPLPR), Tyr-Val-Leu-Leu-Pro-Ser-Pro-Lys (YVLLPSPK), Lys-Val-Pro-Pro-Leu-Leu-Tyr (KVPPLLY), and combinations thereof.

In various embodiments, one or more of the following walnut peptides are preferred:

Peptide I
Thr-Trp-Leu-Pro-Leu-Pro-Arg (TWLPLPR),
Peptide II
Tyr-Val-Leu-Leu-Pro-Ser-Pro-Lys (YVLLPSPK),
and/or
Peptide III
Lys-Val-Pro-Pro-Leu-Leu-Tyr (KVPPLLY).

As already mentioned, the bioactive walnut peptides are particularly useful for topical application to the skin. Accordingly, the instant disclosure is drawn to the use of walnut peptides in methods for treating the skin. In various embodiments, the one or more walnut peptides are applied to the skin in a pharmaceutical or cosmetic composition, which typically includes a physiologically acceptable carrier, for example, water and optionally water-soluble solvents. The pharmaceutical or cosmetic composition includes an amount of the one or more bioactive walnut peptides sufficient to ensure a therapeutically effective amount of the one or more walnut peptides is administered to the skin during use.

The bioactive walnut peptides are useful for treating inflammation of the skin. For example, the bioactive walnut peptides surprisingly and advantageously reduce, treat, or prevent pro-inflammatory cytokines in the skin. Further, the bioactive walnut peptides are useful in methods for treating conditions, diseases, or disorders of the skin such as psoriasis, dermatitis, atopic dermatitis, allergic dermatitis, eczema, spongiosis, edema, hereditary ichthyosis, senile xerosis, palmar hyperkeratosis, plantar hyperkeratosis, cuts, bruises, pore size, skin cancer, healing or reepithelization disorders, keloids, hypertrophic scars, cellulitis, orange peel skin, elastosis, actinic elastosis, keratosis, rosacea, telangiectasia, couperosis, or combinations thereof.

In various embodiments, use of two or more bioactive walnut peptides is preferred. The two or more bioactive walnut peptides may have similar activities or may provide different activities that benefit the skin. In further embodiments, use of three or more bioactive walnut peptides is preferred. The use of multiple bioactive peptides allows for the modification of more than one physiological mechanism in the treatment of skin. For example, one or more bioactive walnut peptide may be useful for preventing and/or treating age-related inflammation while another bioactive walnut peptide may be useful for stimulating fibroblasts that produce collagen and elastin to clarify, thicken, and tighten skin. Moreover, combinations of bioactive walnut peptides can interact synergistically and benefits beyond the sum of the peptides' individual contributions. For example, the synergistic activity of a combination can be at least 5%, at least 10%, or at least 25% greater than the sum of the individual activities of the corresponding amounts of the bioactive walnut peptides.

The one or more bioactive walnut peptides are often incorporated into a pharmaceutical or cosmetic composition for application to the skin. Pharmaceutical and cosmetic composition typically include one or more bioactive walnut peptides and one or more physiologically acceptable carrier, for example water. Nonlimiting examples of physiologically acceptable carriers include water, water soluble solvents such as alcohols, polyols, and glycols, fatty compound such as oils, triglycerides, fatty acids, fatty alcohols, and the like. Pharmaceutical and cosmetic compositions include lotions, creams, serums, sprays, emulsions, gels, powders, dispersions, ointments, sticks, pastes, and foams.

### BRIEF DESCRIPTION OF THE DRAWINGS

Implementation of the present technology is described, by way of example only, with reference to the attached figures, wherein:

FIG. 1 is a heatmap showing differentially expressed genes in adult human epidermal keratinocytes treated with walnut peptides;

FIG. 2 is a heatmap showing differentially expressed genes in reconstructed human epidermis treated with walnut peptides;

FIG. 3 shows histological analysis of skin samples, including a control skin sample, a skin sample treated with microneedle, and skin samples treated with microneedle and walnut peptides; and

FIG. 4 shows histological analysis of skin samples, including a control skin sample, a skin sample treated with a cell stimulation cocktail (CSC), and skin samples treated with CSC and walnut peptides.

The various aspects of the disclosure are not limited to the results, arrangements, and representations shown in the drawings.

### DETAILED DESCRIPTION OF THE DISCLOSURE

The instant disclosure is drawn to bioactive walnut peptides and their topical use for treating skin. Bioactive walnut peptides include two to several dozen amino acids connected to one another through peptide bonds. Their molecular weight is generally less than 6000 Da, preferably less than 3,000 Da, and more preferably less than 1,000 Da. The term “peptide” in accordance with the present disclosure is a compound that includes an uninterrupted sequence of at least two amino acids within its structure and has a maximum of about 50 amino acid. The terms “di-peptide” or “dipeptide” as used herein refer to a compound that includes an uninterrupted sequence of two amino acids within its structure. The terms “tri-peptide” or “tripeptide” as used herein refer to a compound that includes an uninterrupted sequence of three amino acids within its structure. As used herein, a “tetra-peptide” or “tetrapeptide” is a compound that includes an uninterrupted sequence of four amino acids within its structure. These amino acids are indicated herein using a traditional one letter convention from left (N-terminal end) to right (C-terminal end). In this nomenclature, G is glycine, H is histidine, K is lysine, E is glutamic acid, and the like, according to well known and accepted nomenclature in the art.

A “bioactive” peptide for purposes of the instant disclosure has a minimum of 2 or 3 amino acid residues up to about 20 amino acid residues in length and has a measurable physiological effect on skin cells. Bioactive peptides include amino acids joined by covalent bonds, also referred to as amide or peptide bonds, whereas proteins are polypeptides with a greater molecular weight (MW) and typically more than 50 amino acid residues. Bioactive peptides usually display hormone or drug-like activities and are classified based on their mode of action. Many bioactive peptides share some structural features that include, for example, a peptide residue length of 2 to 20 amino acids.

The term “amino acid” as used herein includes and encompasses all naturally occurring amino acids, either in the D- or L-configuration if optically active, and the known non-native, synthetic, and modified amino acids, such as homocysteine, ornithine, norleucine, and p-valine. A list of non-natural amino acids may be found in The Peptides, Vol. 5 (1983), Academic Press, Chapter VI, by D. C. Roberts and F. Vellaccio, which is incorporated herein by reference in its entirety. The amino acids in the peptides of the present invention may be present in their natural L-configuration, unnatural D-configuration, or as a racemic mixture.

As used herein, the term “peptide” shall also refer to salts, deprotected forms, acylated forms of the peptide, deacylated forms of the peptide, enantiomers, diastereomers, racemates, prodrugs and hydrates of the above-mentioned peptide. Diastereomers of the peptide are obtained when the stereochemical or chiral center of one or more amino acids is changed. The enantiomer has the opposite stereochemistry at all chiral centers. In various embodiments, the C-terminal of a peptide is synthesized as an amide to neutralize negative charge created by the C-terminal COOH. This modification can be added to help prevent enzyme degradation.

The term “prodrug” refers to any precursor compound which can generate or to release the above-mentioned peptide under physiological conditions. Such prodrugs are for instance larger peptides which are selectively cleaved to form the peptide of the invention. Further prodrugs are protected amino acids having protecting groups at the carboxylic acid and/or amino group. Suitable protecting groups for amino groups include, for example, the benzyloxycarbonyl, t-butyloxycarbonyl (BOC), formyl, and acetyl or acyl group. Suitable protecting groups for the carboxylic acid group are esters such as benzyl esters or t-butyl esters.

Epidermal impairment can result from acute injury or exposure. Virtually all dysfunctions of the epidermis, whether inborn or acquired, are associated with notable modifications of the permeability barrier. It is particularly evident in dermatoses with an important inflammatory component. In many cases, barrier dysfunction may be at the origin of a skin disease, like it is the case in atopic dermatitis, and contributes to the vicious circle of a given pathology via induction of an inflammatory response. Deficient expression of an epidermal protein filaggrin, due to the loss-of-function gene mutations, has been found responsible for atopic dermatitis occurrence in up to 50% of the northern European cases.

Filaggrin is elaborated in the granular layer keratinocytes and its catabolic processing in the stratum corneum leads to the abundance of hydrophilic amino acids constituting the bulk of so-called natural moisturizing factor (NMF). Absence or a marked reduction of the natural moisturizing factor compromises stratum corneum hydration and, thus, barrier function. Interestingly, the same filaggrin mutations present on both gene alleles result in ichthyosis vulgaris phenotype, most frequently associated with atopy. In the case of ichthyosis, the epidermis must compensate for the leaky barrier by hyperkeratosis. Accumulation of the corneocytes is likely promoted by a particularly low degree of stratum corneum hydration, possibly impeding activity of hydrolytic stratum carenum enzymes. This putative mechanism could overdrive the desquamation-favorable context of serine protease activation due to a more basic (optimal) intracellular pH in the amino acid-deficient tissue. Nano-mechanical and ultrastructural investigations of elastic properties of filaggrin deficient corneocytes demonstrate a significant reduction in the cell stiffness and a delayed degradation of corneodesmosomes, both being potential indicators of stratum corneum functionality. In addition to an alteration of filaggrin expression, atopic dermatitis epidermis also exhibits a significant reduction in key TJ proteins and, most importantly, ceramides, including ceramide EOS.

Regarding the changes to ceramides, their decreased levels and shortening of their acyl chains have been observed in non-involved skin of atopic dermatitis, independent of filaggrin mutations, which may have etiologic significance. Altered ceramide expression levels and both their lamellar and lateral organization correlate with the disease activity. Even more depressed ceramide levels and free sterols have been reported in atopic dermatitis lesions, with concomitant increase of sphingosine and sphinganine-based ceramides.

In psoriasis, inflammatory skin lesions induced by interleukin 23-recruited Th17 lymphocytes are characterized by keratinocyte hyperproliferation and incomplete terminal differentiation leading to inefficient permeability barrier function. Although the immune cell subsets and cytokines involved in atopic dermatitis and psoriasis pathogenesis differ notably, the deleterious vicious circle of barrier disruption/inflammation is still present in the latter. The incomplete terminal differentiation of psoriatic lesional keratinocytes is induced by T-lymphocyte mediated skin inflammation, which has significant impact on the ceramide expression compared to normal or non-involved skin. Like atopic dermatitis, in psoriasis lesions, ceramide species show shorter fatty acid chains. Clinical observations of improvement of psoriasis vulgaris lesions under simple occlusion and of atopic dermatitis lesions with topical emollient therapy alone clearly indicate that restoration of or compensation for the stratum corneum barrier helps to interrupt the vicious circle of pathogenic self-propagation.

To perform its protective functions, epidermis must adapt continuously to the changes in environmental conditions. These encompass climate/season-related factors such as relative humidity, ambient temperature, and sun exposure, as well as environmental aggressions due to the wide-spread use of chemicals, presence of atmospheric pollutants and changes in the composition and importance of skin surface microbiota, the latter being largely related to the aforementioned factors.

Moisture influences stratum corneum turnover by changing the rate of corneocyte desquamation. Indeed, it promotes a rapid rise in the stratum corneum pH, resulting in an increase of activity of kallikreins, the major stratum corneum serine proteases involved in desquamation. Also, water exposure facilitates accessibility of corneodesmosomes to the proteolytic enzymes, which stay otherwise encased within the largely hydrophobic extracellular spaces, and thus promotes release of the cells at the skin surface. Conversely, there is an observed persistence of corneodesmosomes in the outer stratum corneum of xerotic winter skin compared to normal skin. Moreover, cold and dry weather are known to increase the prevalence and risk of flares in patients with atopic dermatitis.

Environmental factors causing impairment of skin barrier function include exposure to irritants and allergens. In the industrialized societies, the skin barrier is affected by the everyday use of detergents and disinfectants, in combination with the deleterious action of atmospheric pollutants that vary with geographic location and source. These pollutants contain solid and liquid particles suspended in the air and various gases such as ozone, nitrogen oxides, volatile organic compounds, and carbon monoxide. Particles vary in number, size, shape, surface area, and chemical composition, while both particles and gases may vary in solubility and toxicity. Occupational factors also play a role since they increase the risks in specific subpopulations. In health care professions, the extensive use of gloves results in occlusion, which significantly worsens the negative effect on skin barrier function of detergents/soaps. The published data indicate that a dose-response relationship is important with respect to duration of occlusion. This is particularly relevant for workplaces where shifting between wearing of gloves and hand washing is common.

The instant disclosure includes methods for improving barrier function of skin. Such methods include treating skin by topical application of a therapeutically effective amount of one or more walnut peptides to the skin. In various embodiments, the one or more bioactive walnut peptides have a molecular weight of less than 10,000 Da. In further embodiments, the one or more bioactive walnut peptides have a molecular weight of less than 8,000 Da, less than 6,000 Da, less than 5,000 Da, less than 4,000 Da, less than 3,000 Da, less than 2,000 Da, less than 1,000 Da, and even less than 500 Da. Preferably, the one or more bioactive walnut peptides have a molecular weight of less than 5,000 Da, more preferably less than 2,000 Da, and even more preferably less than 1,000 Da.

The number of amino acid residues in the one or more bioactive walnut peptides will vary and can be limited based on the molecular weights described above. Nonetheless, in various embodiments, the one or more bioactive walnut peptides comprise from 2 to about 50 amino acid residues. In further embodiments, the one or more bioactive peptides have from 2 to about 25 amino acid residues, from about 2 to about 20 amino acid residues, about 2 to about 18 amino acid residues, about 2 to about 15 amino acid residues, about 2 to about 12 amino acid residues, about 2 to about 10 amino acid residues, about 3 to about 25 amino acid residues, about 3 to about 20 amino acid residues, about 3 to about 18 amino acid residues, about 3 to about 18 amino acid residues, about 3 to about 15 amino acid residues, about 3 to about 12 amino acid residues, or about 3 to about 10 amino acid residues. Preferably, the one or more bioactive peptides comprise 2 to 20 amino acid residues, more preferably 2 to about 15 amino acid residues, even more preferably 3 to about 12 amino acid residues.

In various embodiments, the one or more bioactive walnut peptides include one or more amino acid residues selected from leucine, proline, and combinations thereof. In a further embodiments, the one or more bioactive walnut peptides include three or more amino acid residues selected from leucine, proline, or combination thereof. Nonlimiting examples of bioactive of three or more amino acid residues selected from leucine, proline, and combinations thereof include Leu-Pro-Leu, Leu-Leu-Pro, Pro-Pro-Leu, and Pro-Leu-Pro. For example, the one or more bioactive walnut peptides may comprise an amino acid sequence selected from Thr-Trp-Leu-Pro-Leu-Pro-Arg, Tyr-Val-Leu-Leu-Pro-Ser-Pro-Lys, or Lys-Val-Pro-Pro-Leu-Leu-Tyr. As already mentioned, the bioactive walnut peptides include salts, deprotected forms, acylated forms, deacylated forms, enantiomers, diastereomers, racemates, prodrugs, and hydrates of a particular amino acid sequence.

In a preferred embodiment at least one, and preferably all, of the one or more bioactive walnut peptides are selected from Peptide I, Peptide II, and Peptide III including salts, deprotected forms, acylated forms, deacylated forms, enantiomers, diastereomers, racemates, prodrugs, and hydrates thereof.

Peptide I
TWLPLPR (Thr-Trp-Leu-Pro-Leu-Pro-Arg),
Peptide II
YVLLPSPK (Tyr-Val-Leu-Leu-Pro-Ser-Pro-Lys),
and
Peptide III
KVPPLLY (Lys-Val-Pro-Pro-Leu-Leu-Tyr).

In various embodiments, use of two or more bioactive walnut peptides is desirable. The two or more bioactive walnut peptides may share similar activities or may provide different activities that benefit the skin. In further embodiments, use of three or more bioactive walnut peptides is preferred. The use of multiple bioactive peptides allows for reliance on more than one physiological mechanism for the treatment of skin. For example, one or more bioactive walnut peptide may be useful for preventing and/or inflammation while another bioactive walnut peptide may be useful for stimulating fibroblasts that produce collagen and elastin to clarify, thicken, and tighten skin. Moreover, as shown later, combinations of bioactive walnut peptides interact synergistically to provide benefits beyond the sum of the peptides' individual contributions. For example, the synergistic activity of a combination of bioactive walnut peptides can be at least 5%, preferably at least 10%, more preferably at least 25% greater than the sum of the activity of the corresponding individual amounts of the bioactive walnut peptides.

Combinations of Peptide I, Peptide II, and Peptide III are particularly useful. For example, a combination of Peptide I and Peptide II, a combination of Peptide I and Peptide III, a combination of Peptide II and Peptide III, or a combination of Peptide I, Peptide II, and Peptide III can be used. The peptides in the combinations can be included in various weight ratios to one another as described below.

Peptide I and Peptide II may be used together in a weight ratio of about 1:10 to about 10:1. In further embodiments, Peptide I and Peptide II may be used together in a weight ratio of about 8:1 to about 1:8, about 5:1 to about 1:5, about 4:1 to about 1:4, about 3:1 to about 1:3, about 2:1 to about 1:2, or about 1:1.

Peptide I and Peptide III may be used together in a weight ratio of about 1:10 to about 10:1. In further embodiments, Peptide I and Peptide III may be used together in a weight ratio of about 8:1 to about 1:8, about 5:1 to about 1:5, about 4:1 to about 1:4, about 3:1 to about 1:3, about 2:1 to about 1:2, or about 1:1.

Peptide II and Peptide III may be used together in a weight ratio of about 1:10 to about 10:1. In further embodiments, Peptide II and Peptide III may be used together in a weight ratio of about 8:1 to about 1:8, about 5:1 to about 1:5, about 4:1 to about 1:4, about 3:1 to about 1:3, about 2:1 to about 1:2, or about 1:1.

In various embodiments, the instant disclosure relates to use of one or more walnut peptides or a pharmaceutical or cosmetic composition comprising the one or more walnut peptides for improving skin barrier function or reepithelization of the skin. Accordingly, the disclosure encompasses methods for improving skin barrier function or reepithelization of the skin comprising applying a therapeutically effective amount of one or more walnut peptides or a composition comprising them to skin, including comprises skin and uncompromised skin.

In various embodiments, the instant disclosure relates to use of one or more walnut peptides or a pharmaceutical or cosmetic composition comprising the one or more walnut peptides for treating or repairing damaged skin. Accordingly, the disclosure encompasses methods for treating or repairing damaged skin, comprising applying a therapeutically effective amount of one or more walnut peptides or a composition comprising them to skin in need thereof. In further embodiments, the damaged skin has been physically damaged, chemically damaged, environmentally damaged, sun damaged, or damaged due to disease.

In yet further embodiments, the disclosure relates to use of one or more walnut peptides or a pharmaceutical or cosmetic composition comprising the one or more walnut peptides for wound healing. For example, the disclosure is drawn to methods for improving wound healing and to methods for treating or improving the look of skin as a result of the wound healing properties imparted by the walnut peptides and compositions comprising them. Such methods include: (i) damaging skin tissue using a chemical, a laser, or physical force; and (ii) applying a therapeutically effective amount of one or more bioactive walnut peptides to the damaged skin. Nonlimiting examples of cosmetic procedures that damage skin includes an ablative laser procedure, a nonablative laser procedure, a microneedle procedure, a cryotherapy procedure, a radiofrequency microneedle procedure, dermabrasion, a chemical peel, an exfoliant, or a mechanical or energy-based device, or any chemical procedures that damage the skin (e.g., chemical peels, etc.).

In various embodiments, the instant disclosure relates to use of one or more walnut peptides or a pharmaceutical or cosmetic composition comprising the one or more walnut peptides for improving skin barrier function. Accordingly, the disclosure encompasses methods for improving skin barrier function, comprising applying a therapeutically effective amount of one or more walnut peptides or a composition comprising them to skin in need thereof. In various embodiment, the skin barrier function of healthy, uncompromised skin is improved. In other embodiments, the skin barrier function of damaged skin is improved.

In various embodiments, the instant disclosure relates to use of one or more walnut peptides or a pharmaceutical or cosmetic composition comprising the one or more walnut peptides for treating dry or itchy skin. Accordingly, the disclosure encompasses methods for treating dry or itchy skin, comprising applying a therapeutically effective amount of one or more walnut peptides or a composition comprising them to skin in need thereof.

In various embodiments, the instant disclosure relates to use of one or more walnut peptides or a pharmaceutical or cosmetic composition comprising the one or more walnut peptides for treating a condition, disease, or disorder of the skin selected from psoriasis, dermatitis, atopic dermatitis, allergic dermatitis, eczema, spongiosis, edema, hereditary ichthyosis, senile xerosis, palmar hyperkeratosis, plantar hyperkeratosis, cuts, bruises, pore size, skin cancer, healing or reepithelization disorders, keloids, hypertrophic scars, cellulitis, orange peel skin, elastosis, actinic elastosis, keratosis, rosacea, telangiectasia, couperosis, or combinations thereof. According, the disclosure encompasses methods for treating a condition, disease, or disorder of the skin selected from psoriasis, dermatitis, atopic dermatitis, allergic dermatitis, eczema, spongiosis, edema, hereditary ichthyosis, senile xerosis, palmar hyperkeratosis, plantar hyperkeratosis, cuts, bruises, pore size, skin cancer, healing or reepithelization disorders, keloids, hypertrophic scars, cellulitis, orange peel skin, elastosis, actinic elastosis, keratosis, rosacea, telangiectasia, couperosis, or combinations thereof, comprising applying a therapeutically effective amount of one or more walnut peptides or a composition comprising them to skin in need thereof.

In further embodiments the instant disclosure relates to use of one or more walnut peptides or a pharmaceutical or cosmetic composition comprising the one or more walnut peptides for treating skin suffering from inflammation or an inflammatory disorder and methods for treating skin suffering from inflammation or an inflammatory disorder. Nonlimiting examples of inflammatory disorders include atopic dermatitis, psoriasis, or a combination thereof.

In various embodiments, the instant disclosure relates to use of one or more walnut peptides or a pharmaceutical or cosmetic composition comprising the one or more walnut peptides for potentiating terminal differentiation and keratinization of skin cells. Likewise, the disclosure relates to methods for potentiating terminal differentiation and keratinization of skin cells comprising applying a therapeutically effective amount of one or more walnut peptides to skin in need thereof.

In further embodiments, the bioactive walnut peptides are useful in one or more methods for depigmenting the skin, lightening the skin, brightening the skin, treating hyperpigmentation, and treating melasmic skin. Similarly, the bioactive walnut peptides are useful in methods for evening out skin tone. The bioactive walnut peptides or compositions comprising them can be applied to skin identified as having hyperpigmentation, melasmic skin, sunspots, aged spots, discolored spots, skin having uneven skin tone, etc. In particular embodiments there is disclosed is a method of lightening skin or evening skin tone comprising applying one or more bioactive walnut peptides or a cosmetic composition comprising the one or more bioactive walnut peptides to the skin. The method can further comprise identify a person in need of skin lightening or evening skin tone. The methods can further include inhibiting melanogenesis in a skin cell, inhibiting tyrosinase or tyrosinase synthesis in a skin cell, or inhibiting melanin transport to keratinocytes in a skin cell. The one or more bioactive walnut peptides or a cosmetic composition comprising the one or more bioactive walnut peptides can act as an alpha melanin stimulatory hormone antagonist. The one or more bioactive walnut peptides or a cosmetic composition comprising the one or more bioactive walnut peptides can even out pigmentation of the skin. In nonlimiting aspect, lightening skin can include reducing the appearance of an age spot, a skin discoloration, or a freckle by topical application of the composition to skin having an age spot, skin discoloration, a freckle, etc.

Also disclosed is a method of treating hyperpigmentation comprising applying one or more bioactive walnut peptides or a cosmetic composition comprising the one or more bioactive walnut peptides to the skin. The method can also comprise identifying a person in need of treating hyperpigmentation. Additional methods contemplated include methods for reducing the appearance of an age spot, a skin discoloration, or a freckle, reducing or preventing the appearance of fine lines or wrinkles in skin, or increasing the firmness of skin.

In another embodiment there is disclosed methods for reducing the appearance of uneven skin tone comprising topically applying one or more bioactive walnut peptides or a cosmetic composition comprising the one or more bioactive walnut peptides to skin. The uneven skin tone can be caused by discolored skin. The one or more bioactive walnut peptides or a cosmetic composition comprising the one or more bioactive walnut peptides can be applied to discolored skin (e.g., facial skin, arm skin, leg skin, scalp, neck skin, chess skin, abdomen skin, hand skin, etc.). The discolored skin can be an age spot, blotchy skin, a freckle, hyperpigmented skin, skin suffering from melasma, skin that has been over-exposed to sun, etc. The method can also be used to improve a person's skin tone by topical application of the one or more bioactive walnut peptides or a cosmetic composition comprising the one or more bioactive walnut peptides disclosed throughout this specification to skin that has discolored skin. The method can also be used to prevent the appearance of uneven skin tone by topical application of the compositions disclosed throughout this specification to skin that is at risk of developing uneven skin tone. Skin at risk of developing uneven skin tone includes skin that has been over-exposed to sun, pregnant women, people having or at risk of developing melasma, post-inflammatory hyperpigmentation (e.g., darkening of skin after injury to skin such as an acne lesion or a burn). The one or more bioactive walnut peptides or a cosmetic composition comprising the one or more bioactive walnut peptides of the present invention can also be used to lighten skin.

The one or more walnut peptides are often combined with one or more additional skin active agents. Nonlimiting examples of skin active agents include anti-atrophy agents, antioxidants, depigmenting agents, or combinations thereof. More specific but nonlimiting examples include ceramides, alpha hydroxy acids, beta hydroxy acids, vitamin A, vitamin C, vitamin D, vitamin E, niacinamide, caffeine, ferulic acid, salicylic acid, madecassoside, retinoic acid, benzoyl peroxide, or a combination thereof. In a preferred embodiment, the one or more walnut peptides are combined with one or more ceramides, for example, one or more ceramides selected from selected from free ceramides, e.g., Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, Ceramide EOdS, protein bound ceramides, Phytosphingosine, Sphingosine, ceramide precursors, fatty acids, fatty alcohols, cholesterol, cholesterol sulfate, or combinations thereof.

The one or more walnut peptides may be combined with cholesterol.

The one or more walnut peptides may be combined with one or more sphingolipid. Sphingolipids are a class of lipids containing a backbone of sphingoid bases, which are a set of aliphatic amino alcohols that includes sphingosine. The one or more walnut peptides may be combined with sphingosine-1-phosphate.

The one or more bioactive walnut peptides are often incorporated into a pharmaceutical or cosmetic composition for application to the skin. Pharmaceutical and cosmetic compositions typically include one or more bioactive walnut peptides and a physiologically acceptable carrier. A “physiological carrier” as used herein is a carrier that is appropriate and safe for application to the skin of a human. A particularly common physiologically acceptable carriers is water. However, physiologically acceptable carriers can be oil, fats, organic solvents, and the like, provided they are appropriate and safe for application to the skin. Nonlimiting examples of physiologically acceptable carriers include water, water-soluble solvents such as alcohols, polyols, and glycols, fatty compound such as oils, triglycerides, fatty acids, fatty alcohols, petrolatum, and the like. The pharmaceutical and cosmetic compositions of the instant disclosure can be lotions, creams, serums, sprays, emulsions, gels, powders, dispersions, ointments, sticks, pastes, or foams.

Bioactive walnut peptides can be derived by hydrolysis of walnut proteins into small molecular peptides with molecular weights between the molecular weight of individual amino acids and the molecular weights of the proteins. This can be carried out using biological or chemical methods. For example, bioactive peptides can be prepared by enzymatic procedures, fermentation, and with chemical methods.

Bioactive walnut peptides can be synthesized by coupling the carboxyl group or C-terminus of one amino acid to the amino group or N-terminus of another. Due to the possibility of unintended reactions, protecting groups are sometimes necessary. Chemical peptide synthesis starts at the C-terminal end of the peptide and ends at the N-terminus. Peptides can be synthesized either by solid-phase peptide synthesis, by liquid-phase peptide synthesis, or by fragment condensation. In principle, the seemingly simple formation of a peptide bond can be accomplished using all the procedures available in organic chemistry for the synthesis of carboxylic acid amides.

The general process for synthesizing peptides on solid-phase (e.g., resin) starts by attaching the first amino acid, the C-terminal residue, to the resin. To prevent the polymerization of the amino acid, the alpha amino group and the reactive side chains are protected with a temporary protecting group. Once the amino acid is attached to the resin, the resin is filtered and washed to remove byproducts and excess reagents. Next, the N-alpha protecting group is removed in a deprotection process, and the resin is again washed to remove byproducts and excess reagents. Then the next amino acid is coupled to the attached amino acid. This is followed by another washing procedure, which leaves the resin-peptide ready for the next coupling cycle. The cycle is repeated until the peptide sequence is complete. Then typically, all the protecting groups are removed, and the peptide resin is washed, and the peptide is cleaved from the resin.

Enzymatic hydrolysis involves using commercial enzymes to obtain bioactive peptides. The enzymes are responsible for cleaving the peptide bonds established in the protein, thereby releasing the encrypted peptide. For the enzyme to carry out its activities, it is important for the enzyme bind the substrate and continue with enzymatic catalysis. The enzyme has specific active sites containing residues that form temporary bonds with the substrate and residues that catalyze the reaction with the substrate. In this way, binding sites and catalytic sites are formed, respectively. The bonds forming the enzyme-substrate complex are usually hydrogen bonds, hydrophobic bonds, or Van der Waals interactions. Enzymatic hydrolysis can generally be carried out in three ways: (i) under traditional batch conditions; (ii) using immobilized enzymes; or (iii) using ultrafiltration membranes. Numerous proteolytic enzyme are known and include those described, for example, in Cruz-Casas et al., Enzymatic Hydrolysis and Microbial Fermentation: The Most Favorable Biotechnological Methods for the Release of Bioactive Peptides (FOOD CHEM (OXF). 3:100047, Dec. 30, 2021), which is incorporated herein by reference in its entirety.

Microbial fermentation is a biotechnological process through which bioactive peptides can be obtained. This process involves using microorganisms capable of producing proteolytic enzymes with the objective that these enzymes hydrolyze proteins into shorter peptides. The microorganisms generally used are bacteria, fungi, or yeasts, which may be present in the substrate indigenously or added as a starter culture. The microbial fermentation process can be divided into several systems. However, submerged fermentation and solid-state fermentation are the most widely used.

Submerged fermentation uses a culture of microorganisms in a liquid medium, which contains nutrients. This system is suitable for microorganisms with high water level activities, such as bacteria, and offers the advantage that the generated bioactive peptides are easy to purify. Solid-state fermentation uses microbial growth on nutrient-rich solid substrates. It has the advantage of releasing nutrients in a controlled way and is suitable for fungi and microorganisms with fewer moisture requirements.

Bioactive walnut peptides may be produced using any method known to those skilled in the art such as those disclosed in Merrifield, R. B., Solid Phase Peptide Synthesis I., J. AM. CHEM. SOC. 85:2149-2154 (1963); Carpino, L. A. et al., [(9-Fluorenylmethyl) Oxy] Carbonyl (Fmoc) Amino Acid Chlorides: Synthesis, Characterization, And Application To The Rapid Synthesis Of Short Peptides, J. ORG. CHEM. 37:51:3732-3734; Merrifield, R. B. et al., Instrument For Automated Synthesis Of Peptides, ANAL. CHEM. 38:1905-1914 (1966); or Kent, S. B. H. et al., High Yield Chemical Synthesis Of Biologically Active Peptides On An Automated Peptide

Synthesizer Of Novel Design, IN: PEPTIDES 1984 (Ragnarsson U., ed.) Almqvist and Wiksell Int., Stockholm (Sweden), pp. 185-188, which are all incorporated herein by reference in their entirety.

The therapeutically effective amount of the one or more bioactive walnut peptides will vary depending on the bioactive walnut peptide and the combination of bioactive walnut peptides. Nonetheless, in various embodiments, the therapeutically effective amount may be from about 1 μg to about 50 mg (50,000 μg) per cm² of skin. In further embodiments, the therapeutically effective amount of the one or more walnut peptides is from about 1 μg to about 40 mg, about 1 μg to about 30 mg, about 1 to about 20 mg, about 1 μg to about 10 mg, about 1 μg to about 8,000 μg, about 1 μg to about 5,000 μg, about 1 μg to about 2,000 μg, about 1 μg to about 1,000 μg, about 10 μg to about 40 mg, about 10 μg to about 30 mg, about 10 to about 20 mg, about 10 μg to about 10 mg, about 10 μg to about 8,000 μg, about 10 μg to about 5,000 μg, about 10 μg to about 2,000 μg, about 10 μg to about 1,000 μg, about 100 μg to about 50 mg, about 100 μg to about 40 mg, about 100 μg to about 30 mg, about 100 μg to about 20 mg, about 100 μg to about 10 mg, about 100 μg to about 8,000 μg, about 100 μg to about 5,000 μg, about 100 μg to about 2,000 μg, about 100 μg to about 1,000 μg, about 500 μg to about 50 mg, about 500 μg to about 40 mg, about 500 μg to about 30 mg, about 500 μg to about 30 mg, about 500 μg to about 20 mg, about 500 μg to about 10 mg, about 500 to about 8,000 μg, about 500 μg to about 5,000 μg, about 500 μg to about 2,000 μg, or about 500 μg to about 1,000 μg per cm² of skin.

The bioactive walnut peptides are typically formulated with a physiologically acceptable carrier and applied to the skin as a pharmaceutical or cosmetic composition. The amount of the one or more bioactive walnut peptides will vary depending on their activity, use, and interaction with additional bioactive walnut peptides that may optionally be included in the pharmaceutical or cosmetic composition. Nonetheless, in various embodiments, the pharmaceutical or cosmetic composition include about 0.01 to about 10 wt. % of the one or more bioactive walnut peptides, based on the total weight of the pharmaceutical or cosmetic composition.

In further embodiments, the pharmaceutical or cosmetic composition include about 0.01 to about 8 wt. %, about 0.01 to about 5 wt. %, about 0.01 to about 3 wt. %, about 0.01 to about 1 wt. %, about 0.01 to about 0.5 wt. %, about 0.1 to about 10 wt. %, about 0.1 to about 8 wt. %, about 0.1 to about 5 wt. %, about 0.1 to about 3 wt. %, about 0.1 to about 1 wt. %, about 0.5 to about 10 wt. %, about 0.5 to about 8 wt. %, about 0.5 to about 5 wt. %, about 0.5 to about 3 wt. %, about 0.5 to about 2 wt. %, about 1 to about 10 wt. %, about 1 to about 8 wt. %, about 1 to about 5 wt. %, about 1 to about 3 wt. %, or about 0.1 to about 2 wt. % of the one or more bioactive walnut peptides, based on the total weight of the pharmaceutical or cosmetic composition.

Pharmaceutical and cosmetic compositions including the one or more bioactive walnut peptides can be formulated in various forms, for example, lotions, creams, serums, sprays, emulsions, gels, powders, dispersions, ointments, sticks, pastes, and foams. In addition to a physiologically acceptable carrier, the pharmaceutical and cosmetic composition may optionally include one or more of the following:
Desquamating active agents, keratolytic agents and exfoliating agents,
Suspending agents,
Emulsifying agents,
Thickeners, and/or
Antihistamines.

### Skin Active Agents

Nonlimiting examples of skin active agents include rosacea inhibitory agents (e.g., metronidazole, sulfacetamide, sodium sulfacetamide, sulfur, dapson, doxycycline, minocycline, clindamycin, clindamycin phosphate, erythromycin, tetracylclines, azelaic acid, calcium dobesilate, maleic acid, and any compatible combinations thereof); a-adrenergic receptor agonists (e.g., clonidine, amphetamine, doxtroamphetamine, apraclonidine, dipivefrin, α-methyldopa, oxymetazoline, oxymetazoline hydrochloride, methoxamine, metaraminol, medetomidine, dexmedetomidine, ethylnorepinephrine, guanfacine, guanabenz, phenylephrine, phenylephrine hydrochloride, ephedrine, epinine, epinephrine, ethylnorepinephrine, levarterenol, lofexidine, norepinephrine, norphenylephrine, norephedrine, phenylpropanolamine, pemoline, propylhexadrine, pseudoephedrine, methamphetamine, α-methylnorepinephrine, methylphenidate, mephentermine, midodrine, mivazerol, moxonidine, desglymidodrine, tetrahydrozoline, tetrahydrozoline hydrochloride, cirazoline, amidephrine, brimonidine, brimonidine tartrate, naphazoline, isoproterenol, xylazine, xylometazoline, and/or tizanidine); chemicals and botanical extracts with vasoconstrictor properties including, but not limited to, corticosteroids, ephedrine, pseudoephedrine, caffeine, and/or escin; ephedra, phedra sinica, hamamelis viginiana, hydrastis canadensis, lycopus virginicus, aspidosperma quebracho, cytisus scoparius, raphanus sativus linn (radish leave extracts), horse chestnut extracts, etc.

The one or more bioactive walnut peptides are often combined with one or more additional skin active agents. Nonlimiting examples of skin active agents include anti-atrophy agents, antioxidants, depigmenting agents, or combinations thereof. More specific but nonlimiting examples include ceramides, Melasyl® (N-[(1,2-dihydro-2-thioxo-3-pyridinyl) carbonyl] glycine; INCI: 2-mercaptonictinoyl glycine), alpha hydroxy acids, beta hydroxy acids, vitamin A, vitamin C, vitamin D, vitamin E, niacinamide, caffeine, ferulic acid, salicylic acid, madecassoside, retinoic acid, benzoyl peroxide, or a combination thereof. In a preferred embodiment, the one or more bioactive walnut peptides are combined with one or more ceramides, for example, one or more ceramides selected from selected from free ceramides, e.g., Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, Ceramide EOdS, protein bound ceramides, Phytosphingosine, Sphingosine, ceramide precursors, fatty acids, fatty alcohols, cholesterol, cholesterol sulfate, or combinations thereof.

In a preferred embodiment, the skin active agents are selected from ceramides, ceramide precursors, free fatty acids, Melasyl® (N-[(1,2-dihydro-2-thioxo-3-pyridinyl) carbonyl] glycine; INCI: 2-mercaptonictinoyl glycine), linoleic/oleic acids, cholesterol, cholesterol sulfate, b-glucan, carob seed extract, eperua falcata extract, amino acids, niacinamide and its derivatives, hyaluronic acid & its derivatives, glycerin, allantoin, squalane, omega fatty acids, or combinations thereof.

Additional nonlimiting examples include chemicals or botanical extracts with anti-inflammatory properties (e.g., corticosteroids (for short term use)), non-steroidal anti-inflammatory drugs, linoleic acid, linolenic acid, bisabolol, glycyrrhetinic acid, glycerin, plant extracts with anti-inflammatory properties (i.e., tea extracts, chamomile extracts), anti-inflammatory interleukins (e.g., II-1ra); isoprenylcystein analogues (i.e., N-acetyl-S-farnesyl-L-cysteine), aromatic aldehydes with anti-inflammatory properties (e.g., 4-ethoxy benzaldehyde), etc., as well as any compatible combinations thereof); chemicals or botanical extracts; chemicals or botanical extracts with anti-microbial properties (e.g., antibiotics including, but not limited to gentamicin, penicillins, cephalosporins, quinolones, ciprofloxacin, and/or novobiocin); chemicals or botanical extracts with anti-fungal properties (e.g., ketoconazole, naftifine hydrochloride, oxiconazole nitrate, sulconazole nitrate, urea, terbinafine hydrochloride, selenium sulfide, etc.); chemicals or botanical extracts with anti-dandruff properties; chemicals or botanical extracts with anti-seborrheic properties; keratolytic agents or botanical extracts with keratolytic properties (i.e., alpha-hydroxy acids; beta-hydroxy acids, poly-hydroxy acids, urea, salicylic acid, etc.); chemicals with astringent properties; serine protease inhibitors; saturated dicarboxylic acids; alpha hydroxy acids (e.g., glycolic acids, lactic acid, malic acid, citric acid, tartaric acid, etc.); beta hydroxy acids (e.g., carnitine, 3-hydroxybutyric acid, 3-hydroxypropionic acid, β-hydroxy β-methylbutyric acid, salicylic acid, etc.).

Additional nonlimiting examples of skin active agents include retinoic acid, tretinoin, isotretinoin, adapalene, retinol, and/or derivatives; benzoyl peroxide; dapsone; kinetin (N6-furfuryladenine) and derivatives (e.g., furfurylaminotetrahydro-pyranyladenine); niacinamide (nicotinamide), and combinatijons thereof.

Additional nonlimiting examples of skin active agents include metronidazole, sulfacetamide, sodium sulfacetamide, sulfur, tetracylines, doxycycline, clindamycin, clindamycin phosphate, erythromycin, and/or minocycline, azelaic acid, calcium dobesilate, caffeine, theobromine, theophylline and/or a derivative thereof (i.e., xanthines), vitamin A, vitamin B1, vitamin B2, vitamin B3, vitamin B5, vitamin B6, vitamin B7, vitamin B9, vitamin B12, vitamin C, vitamin D, vitamin E and vitamin K, creatine, carnitine, and essential fatty acids such as linoleic acid and/or linolenic acid, zinc salts such as, for example, zinc sulfate, zinc chloride, zinc glycinate, zinc gluconate, zinc-histidine, zinc L-2-pyrrolidone-5-carboxylate (zinc PCA), zinc salt of linoleic acid, zinc salt of linolenic acid, zinc salt of azelaic acid, zinc peptides, zinc oxide, copper salts including, but not limited to, copper sulfate, copper chloride, copper glycinate, copper gluconate, copper-histidine, copper L-2-pyrrolidone-5-carboxylate (copper PCA), copper salt of linoleic acid, copper salt of linolenic acid, copper salt of azelaic acid, copper peptides,

Nonlimiting examples of skin active agents include madecassoside, retinoic acid, benzoyl peroxide, sulfur, vitamin B6 (pyridoxine or) chloride, selenium, samphire, cinnamon extract blends, zinc gluconate, zinc pyrrolidonecarboxylate (or zinc pidolate), zinc lactate, zinc aspartate, zinc carboxylate, zinc salicylate, zinc cysteate, copper and copper pidolate as Cuivridone Solabia, extracts from plants of Arnica montana, Cinchona succirubra, Eugenia caryophyllata, Humulus lupulus, Hypericum perforatum, Mentha pipenta, Rosmarinus officinalis, Salvia officinalis and Thymus vulgaris, extracts of meadowsweet (Spiraea ulmaria), and mixtures thereof.

In various embodiments, one or more skin active agents may include adenosine, 2-[4-(2-hydroxyethyl) piperazin-1-yl]ethanesulfonic acid (HEPES), hyaluronic acid, lanolin, citric acid, malic acid, lactic acid, tartaric acid, salicylic acid, vitamin C, a vitamin, a retinoid, retinal, retinoic acid, a carotenoid, an amino acid, a protein, an enzyme, and a coenzyme. In some cases the active ingredient is adenosine.

In one embodiment the composition includes one or more skin active agents selected from humectants and moisturizing ingredients, depigmenting agent, or an agent that treats oily skin.

Humectants and moisturizing ingredients may be in particular glycerol and its derivatives, urea and its derivatives, especially Hydrovance marketed by National Starch, lactic acid, hyaluronic acid, AHA, BHA, sodium pidolate, xylitol, serine, sodium lactate, ectoin and its derivatives, chitosan and its derivatives, collagen, plankton, an extract of Imperata cylindra sold under the name Moist 24 by Sederma, homopolymers of acrylic acid as Lipidure-HM of NOF Corporation, beta-glucan and in particular sodium carboxymethyl beta-glucan Mibelle-AG-Biochemistry, a mixture of oils passionflower, apricot, corn, and rice bran sold by Nestle under the name NutraLipids, a C-glycoside derivatives, in particular the C-13-D-xylopyranoside-2-hydroxypropane in the form of a solution at 30% by weight of active material in a water/propylene glycol mixture (60/40 wt %) as the product produced by the company Chimex under the trade name “Mexoryl SBB”, a rose hip oil marketed by Nestle, a micro-algae extract Prophyridium cruentum enriched with zinc, marketed under the name by Vincience Algualane Zinc spheres of collagen and chondroitin sulfate of marine origin (Atelocollagen) sold by the company Engelhard Lyon under the name Marine Filling Spheres, hyaluronic acid spheres such as those marketed by Engelhard Lyon, and arginine.

Depigmenting agents include vitamin C and its derivatives and especially vitamin CG, CP and 3-O ethyl vitamin C, alpha and beta arbutin, ferulic acid, lucinol and its derivatives, kojic acid, resorcinol and derivatives thereof, tranexamic acid and derivatives thereof, gentisic acid, homogentisic, methyl gentisate or homogentisate, dioic acid, D pantheteine calcium sulphonate, lipoic acid, ellagic acid, vitamin B3, linoleic acid and its derivatives, Melasyl® (N-[(1,2-dihydro-2-thioxo-3-pyridinyl) carbonyl] glycine; INCI: 2-mercaptonictinoyl glycine), ceramides and their counterparts, derived from plants such as chamomile, bearberry, the aloe family (vera, ferox, bardensis), mulberry, skullcap, a water kiwi fruit (Actinidia chinensis) marketed by Gattefosse, an extract of Paeonia suffruticosa root, such as that sold by Ichimaru Pharcos under the name Liquid Botanpi Be an extract of brown sugar (Saccharum officinarum) such as molasses extract marketed by Taiyo Kagaku under the name Liquid Molasses, without this list being exhaustive. Particular depigmenting agents include vitamin C and its derivatives and especially vitamin CG, CP and 3-O ethyl vitamin C, alpha and beta arbutin, ferulic acid, kojic acid, resorcinol and derivatives, D pantheteine calcium sulfonate, lipoic acid, ellagic acid, vitamin B3, a water kiwi fruit (Actinidia chinensis) marketed by Gattefosse, an extract of Paeonia suffruticosa root, such as that sold by the company Ichimaru Pharcos under the name Botanpi Liquid B.

Useful skin active agents include adenosine and its derivatives and retinol and its derivatives such as retinol palmitate, ascorbic acid and its derivatives such as magnesium ascorbyl phosphate and ascorbyl glucoside; tocopherol and derivatives thereof such as tocopheryl acetate, nicotinic acid and its precursors such as nicotinamide; ubiquinone; glutathione and precursors thereof such as L-2-oxothiazolidine-4-carboxylic acid, the compounds C-glycosides and their derivatives as described in particular in EP-1345919, in particular C-beta-D-xylopyranoside-2-hydroxy-propane as described in particular in EP-1345919, plant extracts including sea fennel and extracts of olive leaves, as well as plant and hydrolysates thereof such as rice protein hydrolysates or soybean proteins; algal extracts and in particular laminaria, bacterial extracts, the sapogenins such as diosgenin and extracts of Dioscorea plants, in particular wild yam, comprising: the α-hydroxy acids, f3-hydroxy acids, such as salicylic acid and n-octanoyl-5-salicylic oligopeptides and pseudodipeptides and acyl derivatives thereof, in particular acid {2-[acetyl-(3-trifluoromethyl-phenyl)-amino]-3-methyl-} acetic acid and lipopeptides marketed by the company under the trade names SEDERMA Matrixyl 500 and Matrixyl 3000; lycopene, manganese salts and magnesium salts, especially gluconates, and mixtures thereof.

As adenosine derivatives include especially non-phosphate derivatives of adenosine, such as in particular the 2′-deoxyadenosine, 2′,3′-adenosine isopropoylidene; the toyocamycine, 1-methyladenosine, N-6-methyladenosine; adenosine N-oxide, 6-methylmercaptopurine riboside, and the 6-chloropurine riboside.

Other derivatives include adenosine receptor agonists such as adenosine adenosine phenylisopropyl (“PIA”), 1-methylisoguanosine, N6-cyclohexyladenosine (CHA), N6-cyclopentyladenosine (CPA), 2-chloro-N6-cyclopentyladenosine, 2-chloroadenosine, N6-phenyladenosine, 2-phenylaminoadenosine, MECA, N 6-phenethyladenosine, 2-p-(2-carboxy-ethyl) phenethyl-amino-5′-N-ethylcarboxamido adenosine (CGS-21680), N-ethylcarboxamido-adenosine (NECA), the 5′(N-cyclopropyl)-carboxamidoadenosine, DPMA (PD 129.944) and metrifudil.

In one embodiment the composition comprises an active ingredient that addresses oily skin. These actives can be sebo-regulating or antiseborrhoeic agents capable of regulating the activity of sebaceous glands. These include: retinoic acid, benzoyl peroxide, sulfur, vitamin B6 (pyridoxine or) chloride, selenium, samphire—the cinnamon extract blends, tea and octanoylglycine such as—15 Sepicontrol A5 TEA from Seppic—the mixture of cinnamon, sarcosine and octanoylglycine marketed especially by Seppic under the trade name Sepicontrol A5—zinc salts such as zinc gluconate, zinc pyrrolidonecarboxylate (or zinc pidolate), zinc lactate, zinc aspartate, zinc carboxylate, zinc salicylate 20, zinc cysteate;—derivatives particularly copper and copper pidolate as Cuivridone Solabia—extracts from plants of Arnica montana, Cinchona succirubra, Eugenia caryophyllata, Humulus lupulus, Hypericum perforatum, Mentha pipenta 25 Rosmarinus officinalis, Salvia officinalis and Thymus vulgaris, all marketed for example by Maruzen—extracts of meadowsweet (Spiraea ulmaria), such as that sold under the name Sebonormine by Silab—extracts of the alga Laminaria saccharina, such as that sold under the 30 name Phlorogine by Biotechmarine—the root extracts of burnet mixtures (Sanguisorba officinalis/Poterium officinale), rhizomes of ginger (Zingiber officinalis) and cinnamon bark (Cinnamomum cassia), such as that sold under the name Sebustop by Solabia—extracts of flaxseed such as that sold under the name Linumine by Lucas Meyer—Phellodendron extracts such as those sold under the name Phellodendron extract BG by Maruzen or Oubaku liquid B by Ichimaru Pharcos—of argan oil mixtures extract of Serenoa serrulata (saw palmetto) extract and sesame seeds such as that sold under the name Regu SEB by Pentapharm—mixtures of extracts of willowherb, of Terminalia chebula, nasturtium and of bioavailable zinc (microalgae), such as that sold under the name Seborilys Green Tech;—extracts of Pygeum afrianum such as that sold under the name Pygeum afrianum sterolic lipid extract by Euromed—extracts of Serenoa serrulata such as those sold under the name Viapure Sabal by Actives International, and those sold by the company Euromed—of extracts of plantain blends, Berberis aquifolium and sodium salicylate 20 such as that sold under the name Seboclear Rahn—extract of clove as that sold under the name Clove extract powder by Maruzen—argan oil such as that sold under the name Lipofructyl Laboratories Serobiologiques; 25—lactic protein filtrates, such as that sold under the name Normaseb by Sederma—the seaweed laminaria extracts, such as that sold under the name Laminarghane by Biotechmarine—oligosaccharides seaweed Laminaria digitata, such as that sold under the name Phycosaccharide 30 AC by the company Codif—extracts of sugar cane such as that sold under the name Policosanol by the company Sabinsa, the sulfonated shale oil, such as that sold under the name Ichtyol Pale by Ichthyol—extracts of ‘meadowsweet (Spiraea ulmaria) such as that sold under the name Cytobiol Ulmaire by societeLibiol—sebacic acid, especially sold in the form of a sodium polyacrylate gel under the name Sebosoft by Sederma—glucomannans extracted from konjac tuber and modified with alkylsulfonate chains such as that sold under the name Biopol Beta by Arch Chemical—extracts of Sophora angustifolia, such as those sold under the name Sophora powder or Sophora extract by Bioland—extracts of cinchona bark succirubra such as that sold under the name Red Bark HS by Alban Muller—extracts of Quillaja saponaria such as that sold under the name 15 Panama wood HS by Alban Muller—glycine grafted onto an undecylenic chain, such as that sold under the name Lipacide UG OR by SEPPIC—the mixture of oleanolic acid and nordihydroguaiaretic acid, such as that sold under the form of a gel under the name AC.Net by Sederma; 20—phthalimidoperoxyhexanoic acid—citrate tri (C12-C13) sold under the name COSMACOL ECI by Sasol; trialkyl citrate (C14-C15) sold under the name COSMACOL ECL by Sasol—10-hydroxydecanoic acid, including mixtures acid-hydroxydecanoic October 25, sebacic acid and 1,10-decandiol such as that sold under the name Acnacidol BG by Vincience and mixtures thereof.

Other skin active agents useful herein include those selected from N-acetyl D-glucosamine, panthenol (e.g., DL panthenol available from Alps Pharmaceutical Inc.), tocopheryl nicotinate, benzoyl peroxide, 3-hydroxy benzoic acid, flavonoids (e.g., flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate: DL-a-tocopheryl acetate available from Eisai), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

In various embodiments, the total amount of skin active agents in the pharmaceutical or cosmetic composition of the disclosure, other than the one or more bioactive walnut peptides may be included in an amount greater than zero to about 9 wt. %, greater than zero to about 8 wt. %, greater than zero to about 7 wt. %, greater than zero to about 6 wt. %, greater than zero to about 5 wt. %, greater than zero to about 4 wt. %, greater than zero to about 3 wt. %, greater than zero to about 2 wt. %; about 10 ppm to about 10 wt. % (100,000 ppm), about 10 ppm to about 5 wt. % (50,000 ppm), about 10 ppm to about 2.5 wt. % (25,000 ppm), about 10 ppm to about 1 wt. % (10,000 ppm), about 10 ppm to about 0.5 wt. % (5,000 ppm), about 10 ppm to about 0.3 wt. % (3,000 ppm), about 10 ppm to about 0.2 wt. % (2,000 ppm), about 10 ppm to about 0.1 wt. % (1,000 ppm), about 10 ppm to 500 ppm; about 0.1 to about 10 wt. %, about 0.1 to about 5 wt. %, about 0.1 to about 2.5 wt. %, about 0.1 to about 1 wt. %, about 0.1 to about 0.5 wt. %; about 1 to about 10 wt. %, about 1 to about 8 wt. %, about 1 to about 6 wt. %, about 1 to about 5 wt. %, about 1 to about 4 wt. %, about 1 to about 3 wt. %; about 2 to about 10 wt. %, about 2 to about 8 wt. %, about 2 to about 6 wt. %, about 2 to about 5 wt. %, about 2 to about 4 wt. %; about 3 to about 10 wt. %, about 3 to about 8 wt. %, about 3 to about 6 wt. %, about 3 to about 5 wt. %; about 4 to about 10 wt. %, about 4 to about 8 wt. %, or about 4 to about 6 wt. %, based on the total weight of the pharmaceutical or cosmetic composition.

### Emollients

One or more emollients may also optionally be included in the pharmaceutical or cosmetic composition described herein. An emollient generally refers to an ingredient that can help maintain a soft, smooth, and supple skin appearance. Emollients generally remain on the skin surface or in the stratum corneum and act as a moisturizer or lubricant and reduce delamination. Nonlimiting examples of emollients include acetylarginine, acetylated lanolin, algal extract, polyethylene glycol-6 esters from apricot kernel oil, polyethylene glycol-11 esters from avocado oil, bis-polyethylene glycol-4 dimethicone, butoxyethyl stearate, glycol esters, alkyl glycol ethers esters, cetyl laurate, coconut polyethylene glycol-10 esters, alkyl tartrates, diethyl sebacate, dihydrocholesteryl butyrate, dimethiconol, dimyristyl tartrate, distearare-5 lauroylg utamat, etilavokadat, ethylhexyl myristate, glyceryl isostearate, glyceryl oleate, geksildetsilstearat, geksilizostearat, hydrogenated palm glycerides, hydrogenated soy glycerides, hydrogenated glycerides of fat izostearilneopentanoat, isostearyl palmitate, izotridetsilizononanoat, laureth-2 acetate, lauryl polyglyceryl-6 cetearyl glycol ether, metilglyutset-20 benzoate, mineral oil, palm oil, coconut oil, miret-3 palmitate, octyldecanol, octyldodecanol, Odontella aurita oil, 2-oleamido-1,3 octadecandiol, pal commercial glycerides, glycerides of polyethylene glycols avocado, polyethylene glycol castor oil, copolymer of polyethylene glycol-2/dodecyl glycol, glycerides of polyethylene glycol shea butter, phytol, raffinose, stearyl citrate, glycerides of sunflower seed oil, non-ointment, small tocopheryl glucoside.

### Suspending Agents

The pharmaceutical or cosmetic composition of the present invention may optionally include one or more suspending agents, preferably in a concentration effective to suspend the water-insoluble material in a dispersed form in the compositions or to modify the viscosity of the composition. Such concentrations will vary. Nonetheless, in certain embodiments, the pharmaceutical and cosmetic composition includes from about 0.1% to about 10%, more preferably from about 0.25% to about 5.0% of the one or more suspending agents, based on the total weight of the composition. Nonlimiting examples include vinyl polymers, such as cross-linked acrylic acid polymers, called carbomer, cellulose derivatives and modified cellulose polymers such as methyl cellulose, ethyl cellulose, nitrocellulose, carboxymethyl cellulose, crystalline cellulose, cellulose powder, polyvinylpyrrolidone, polyvinyl alcohol, guar gum, hydroxypropyl guar gum, gum Arabic, galactan, locust bean gum, pectin, agar, starch (rice, corn, potato, wheat), algal colloids (algae extract), microbiological polymers such as dextran, succinoglycan, pullulan, starch-based polymers such as carboxymethyl starch, methyl starch, alginic acid polymers such as sodium alginate, alginic acid propylene glycol esters, acrylate polymers such as sodium polyacrylate, polyacrylate, polyacrylamide, polyethyleneimine and inorganic minutes water soluble material such as bentonite, aluminum magnesium silicate, laponite, hectonite, and anhydrous silicic acid.

Other optional suspending agents include crystalline suspending agents that can be resolved into acyl derivatives, long chain amine oxides, long chain acyl derivatives, and mixtures thereof. Said preferred suspending agents include fatty acid ethylene glycol esters, fatty acid alkanolamides, long chain fatty acid esters (for example, stearyl stearate, cetyl palmitate, etc.); long chain esters of long chain alkanolamides (for example, stearamide diethanolamide distearate, stearamide monoethanol amide stearate); and glyceryl esters (e.g., glyceryl distearate, trihydroxystearin, tribhengen). Other suitable suspending agents include primary amines containing a fatty alkyl fragment containing at least about 16 carbon atoms, examples of which include palmitamine or stearamine, and secondary amines containing two fatty alkyl fragments, each of which contains at least about 12 carbon atoms examples of which include dipalmitoylamine or di (hydrogenated fat) amine. Other suitable suspending agents include phthalic acid di amide (hydrogenated fat) and a crosslinked maleic anhydride/methyl vinyl ether copolymer.

### Emulsifying Agents

Nonlimiting examples of emulsifying agents include condensation products of alkylene oxides with fatty acids (ie alkylene oxide fatty acid esters), condensation products of alkylene oxides with 2 moles of fatty acids (ie fatty acid alkylene oxide diesters), condensation products alkylene oxides with fatty alcohols (ie fatty alcohol alkylene oxide esters), condensation products of alkylene oxides with both fatty acids and fatty alcohols [i.e. where a portion of the polyalkylene oxide is esterified at one end with a fatty acid and esterified (ie, via an ether bond) at the other end with a fatty alcohol]. Non-limiting examples of non-ionic surfactants derived from said alkylene oxide include cetet-6, cetet-10, cetet-12, cetetaret-6, cetetaret-10, cetetaret-12, stearet-6, stearet-10, stearet-12, stearet-21, PEG-6 stearate, PEG-10 stearate, PEG-100 stearate, PEG-12 stearate, PEG-20 glyceryl stearate, PEG-80 glyceryl tallowat, PEG-10 glyceryl stearate, PEG-30 glyceryl cocoate, PEG-80 glyceryl coco, PEG-200 glyceryl tallowat, PEG-8 dilaurate, PEG-10 distearate and mixtures thereof. Other applicable non-ionic surfactants include polyhydroxyamide fatty acid surfactants. A particularly preferred surfactant corresponding to the above structure is N-methylglucoside coconut alkylamide. Preferred among nonionic surfactants are surfactants selected from the group consisting of stearet-21, ceteareth-20, ceteareth-12, sucrose cocoate, stearet-100, PEG-100 stearate and mixtures thereof. Other non-ionic surfactants suitable for use in this application include sugar esters and polyesters, alkoxylated sugar esters and polyesters, C1-C30 fatty acid esters C1-C30 fatty alcohols, alkoxylated C1-C30 ester derivatives C1-C30 fatty alcohol fatty acids, alkoxylated C1-C30 fatty alcohol esters, polyglyceryl C1-C30 fatty acid esters, C1-C30 polyol esters, C1-C30 polyol esters, alkyl phosphates, polyoxyalkylene fatty ether forfates, fatty acid amides, acylactylates and mixtures thereof. Non-limiting examples of these emulsifiers include: polyethylene glycol 20 sorbitan monolaurate (polysorbate 20), polyethylene glycol 5 soy sterol, stearet-20, cetearet-20, PPG-2 methyl glucose ether distearate, cetet-10, polysorbate 80, cetyl phosphate, cetyl phosphate, cetyl phosphate, cetyl phosphate, cetyl phosphate, polysorbate 60, glyceryl stearate, polyoxyethylene 20 sorbitan triolcat (polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 and their PEG-100. Another group of non-ionic surfactants useful herein is a mixture of fatty acid esters based on a mixture of sorbitan or a sorbitol fatty acid ester and a sucrose fatty acid ester, where the fatty acid in each example is preferably C8-C24, more preferably C10-C20 fatty acid.

### Thickeners

Thickeners suitable for inclusion in the pharmaceutical or cosmetic composition described herein. Nonlimiting examples include acrylamide copolymer, agarose, amylopectin, bentonite, calcium alginate, calcium carboxymethyl cellulose, carbomer, carboxymethylchitin, cellulose gum, dextrin, gelatin, hydrogenated hydroxymethyl hydroxy cellulose hydroxypropyl, hydroxyethyl hydroxypropyl hydroxypropyl, hydroxyethyl hydroxypropyl, hydroxyethyl hydroxypropyl, hydroxyethyl hydroxypropyl, hydroxyethyl hydroxypropyl, hydroxyethyl hydroxypropyl, hydroxypropyl, hydroxypropyl, hydroxypropyl, hydroxypropyl, hydroxypropyl; magnesium alginate, methyl cellulose, microcrystalline cellulose, pectin, various polyethylene glycols, polyacrylic acid, polymethacrylic acid, polyvinyl alcohol, various sexes ipropylene glycols, copolymers of sodium acrylates, sodium carrageenan, xanthan gum and/or yeast beta-glucan, or mixtures thereof.

More generally, carboxylic acid polymers are useful thickeners. Polymers of carboxylic acids are crosslinked compounds containing one or more monomers derived from acrylic acid, substituted acrylic acids and salts and esters of said acrylic acids and substituted acrylic acids, wherein the crosslinking agent contains two or more carbon-carbon double bonds and comes from polyhydric alcohol. Examples of commercially available carboxylic acid polymers useful herein include carbomers, which are homopolymers of acrylic acid crosslinked with sucrose or pentaerythrotol allyl ethers. Carbomers are available as Carbopol® 900 Series from B.F. Goodrich (e.g. Carbopol® 954). In addition, other suitable carboxylic acid based polymeric agents include copolymers of C10-30 alkyl acrylates with one or more monomers of acrylic acid, methacrylic acid or esters of one of its short chains (i.e., C1-4 alcohol), the crosslinking agent is a sucrose or pentaerythritol allyl ether. These copolymers are known as acrylate/C10-30 alkyl acrylate crosspolymers and are commercially available as B.F. Carbopol® 1342, Carbopol® 1382, Pemulen TR-1 and Pemulen TR-2. Goodrich. Examples of preferred thickeners based on carboxylic acid polymers useful in this application include thickeners selected from carbomers, acrylate/C10-30 alkyl acrylate cross-polymers and mixtures thereof.

Moreover, according to certain embodiments, the thickeners are selected from polysaccharides. Nonlimiting examples of polysaccharide thickeners include cellulose, carboxymethyl hydroxyethyl cellulose, cellulose acetate propionate carboxylate, hydroxyethyl cellulose, hydroxyethyl cellulose, hydroxypropyl cellulose, hydroxypropyl methyl cellulose, sodium methyl hydroxyethyl cellulose hydroxyethyl cellulose and hydroxyethyl methyl hydroxyethyl hydroxyethyl cellulose hydroxyethyl methyl hydroxyethyl hydroxyethyl cellulose hydroxyethyl hydroxyethyl cellulose hydroxyethyl methyl hydroxyethyl hydroxyethyl. Alkyl-substituted celluloses are also useful. In these polymers, the hydroxy groups of the cellulosic polymer are hydroxylated (preferably hydroxyethylated or hydroxypropylated) to form hydroxylated cellulose, which is then further modified with a C10-30 straight or branched chain alkyl group via an ether bond. Typically, these polymers are straight chain or branched chain esters of C10-30 alcohols and hydroxyalkyl celluloses. Examples of alkyl groups applicable in this application include groups selected from stearyl, isostearyl, lauryl, myristyl, cetyl, isocetyl, cocoyl (e.g., an alkyl group derived from coconut oil alcohols), palmityl, oleyl, linoleil, linolenyl, recinoleil, behenyl and mixtures thereof. A preferred alkyl hydroxyalkyl cellulose ester is a material called cetyl hydroxyethyl cellulose, which is an ester of cetyl alcohol and hydroxyethyl cellulose, in accordance with the Perfume and Cosmetics and Perfume Association (CTFA). The indicated material is sold under the trade name Natrosol® CS Plus from Aqualon Corporation (Wilmington, Delaware). Further examples can be found in The International Cosmetic Ingredient Dictionary and Handbook, the Cosmetic Bench Reference-Directory of Cosmetic Ingredients, offered by the United States Pharmacopeia (USP) and National Formulary (NF), and other references to cosmetic and pharmaceutical ingredients known in the art. technicians. Other useful polysaccharides include scleroglucans, which are a straight chain (1-3) linked glucose units (1-6), where every three glucose units are linked, a commercially available example of which is Clearogel™ CS11 from Michel Mercier Products Inc. (Mountainside, New Jersey).

Other useful thickeners include those derived from natural sources. Nonlimiting examples include gum arabic, agar, algin, alginic acid, ammonium alginate, amylopectin, calcium alginate, calcium carrageenan, carnitine, carrageenan, dextrin, gelatin, gellan gum, guar gum hydrochloride, gidrohydrochloride, gidrohydrochloride, gidrohydrochloride, hydrochloride hyaluronic acid, hydrated silicon dioxide, hydroxypropylchitosan, hydroxypropyl gum, karaya gum, kelp, fruit tree resin, natto gum, potassium alginate, potassium carr gene, propylene glycol alginate, sclerotium gum, sodium carboxymethyl dextran, dextran, sodium carrageenan, tragacanth gum, xanthan gum and/or mixtures thereof.

In addition, the compositions may optionally contain polyacrylamide polymers, in particular nonionic polyacrylamide polymers, including substituted branched or unbranched polymers. Other polyacrylamide polymers useful herein include multi-block copolymers of acrylamides and substituted acrylamides with acrylic acids and substituted acrylic acids.

### Antihistamines

Antihistamines, also called histamine antagonists, are substances that inhibit the action of histamine by blocking its attachment to histamine receptors or by inhibiting the enzymatic activity of histidine decarboxylase, which catalyzes the conversion of histidine to histamine and the like. Nonlimiting examples of antihistamines are acrivastine, azelastine, brompheniramine, buclizine, bromodiphenhydramine, carbinoxamine, cetirizine, chlorpromazine, cyclizine, chlorpheniramine, chlorodiphenhydramine, cimetidine, clemastine, cyproheptadine, desloratine dimendenine dimendenine dimendenine dimendenine dimendenine dimendenine dimendenine dimendenine dimendenine dimenhydramin dimendenine dimendenine dimendenine dimendenine dimendenine dimendenine dimendenine dimendenine dimendenine dimendenine dimendenine dimendenine dimen dimerdenine dimen dimendenine dimenhydramine ebastine, embramine, famotidine, fexofenadine, lafutidine, levocetirizine, loratadine, meclosine, mirtazapine, nizatidine, olopadadine, orphenadrine, phenindamine, pheniramine, phenyltoloxamine, prometh zine, pyrilamine, quetiapine, ranitidine, roxatidine, rupatadin, tripelennamine, and triprolidine.

### EXAMPLES

Various changes can be made in the above-described compositions and methods without departing from the scope of the invention. Accordingly, it is intended that all disclosure contained in the above description and in the examples given below, shall be interpreted as illustrative and not in a limiting sense.

### Example 1

### Differential Gene Expression

Peptide I, II, and III were investigated for their ability to influence gene expression at concentration at 100 μM abd 500 μM for cells and 250 μM for reconstructed human epidermis (RHE).

### Adult Normal Human Epidermal Keratinocytes

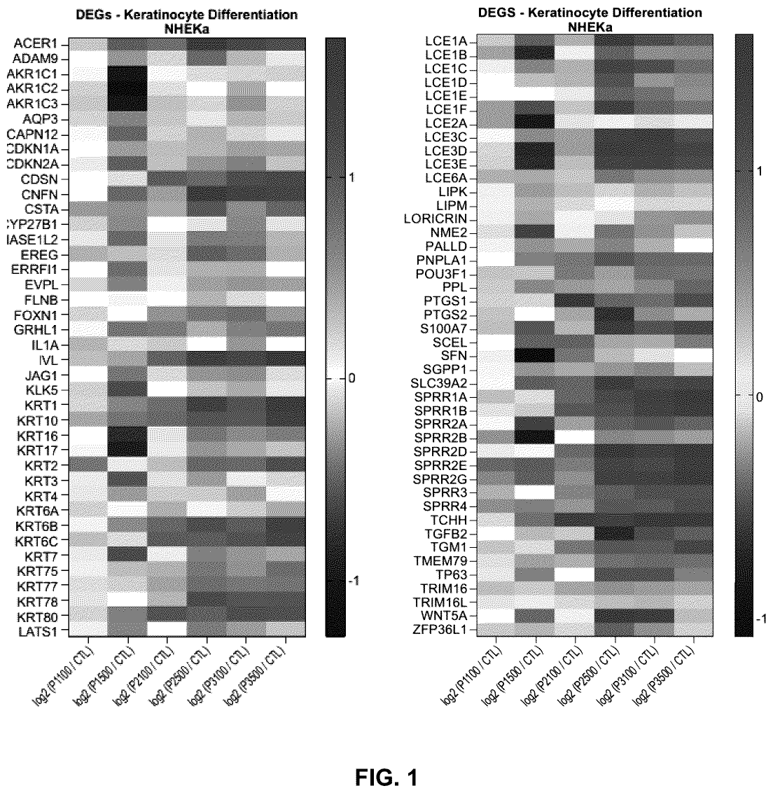

Adult Normal Human Epidermal Keratinocytes (Lot 80819221, Passage 3, ATCC) were expanded and seeded into a 6-well plate at a density of 300,000 cells/well. Cells were grown in Dermal Cell Basal Medium (ATCC PCS-200-030) supplemented with one Keratinocyte Growth Kit (ATCC PCS-200-040). Cells attached overnight and then culture media was aspirated and replaced with media containing either 100 uM or 500 uM of Peptide I, Peptide II, or Peptide III. Cells were cultured for 4 days with treatment with renewal of treatment medium every other day with the exception of weekends. Following the 7-day incubation period, cells were trypsinized and the cell pellet was snap-frozen using liquid nitrogen. Cells then underwent RNA sequencing. The log 2 fold change for each treatment group compared to untreated control samples for differentially expressed genes (DEGs) related to skin barrier were analyzed. Fold change was plotted via heatmap to show upregulation or downregulation of these genes. Microsoft Excel and GraphPad Prism were used for additional analysis and data presentation. The results are shown in the heatmap of FIG. 1. The data show that Peptide II and Peptide III provide a strong benefits to the skin barrier on a cellular level at both concentrations tested, while Peptide I showed an influence at the higher concentration.

### Reconstructed Human Epidermis

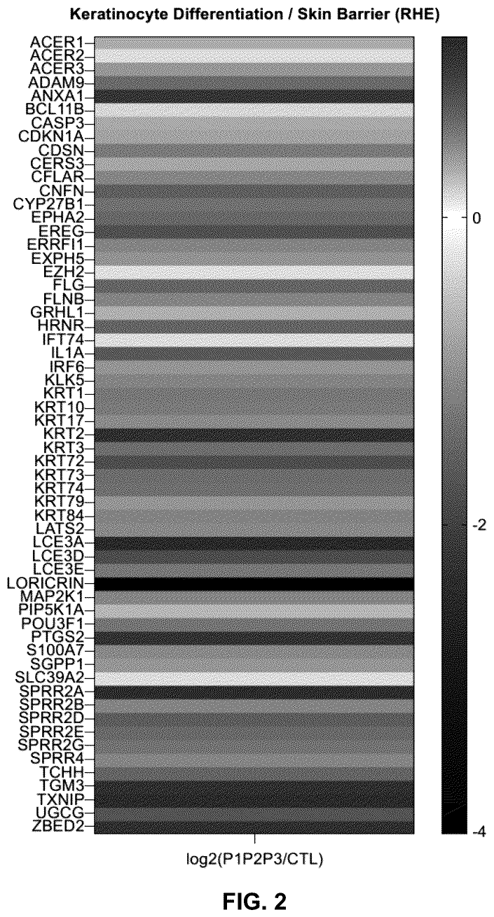

Reconstructed human epidermis (RHE) (EPISKIN/S/13, Episkin) was received and samples were prepared for use. Upon arrival, inserts containing the RHE were removed from the multi-plate and placed in a 12-well plate containing maintenance medium. Tissue serving as untreated control were placed in regular maintenance medium; treatment groups were placed in maintenance medium containing 250 uM of Peptide I, II, and III in combination. Medium was aspirated and replaced every other day with the exception of weekends. RHE was cultured at air-liquid interface at 37 C, 5% CO₂ and saturated humidity for 7 days. Following culture, samples were snap-frozen in liquid nitrogen and sent for RNA sequencing. The log 2 fold change for each treatment group compared to untreated control samples for differentially expressed genes (DEGs) related to skin barrier were analyzed. Fold change was plotted via heatmap to show upregulation or downregulation of these genes. Microsoft Excel and GraphPad Prism were used for additional analysis and data presentation, shown in the heat map of FIG. 2. The data show that a combination of the three peptides provide improved skin barrier function for on RHE.

### Example 2

### Barrier Renewal—Microneedle Ex Vivo Model

Fresh post-abdominoplasty normal human skin samples were acquired from BioIVT Inc. (Westbury, NY). Tissue was defatted and cleaned of blood residue. Tissue was then subjected to treatment using a microneedling pen (36-pin needles, Dr. Pen A6 Cartridges Tips, Dr. Pen Inc., San Jose, CA, USA) with a needle length of 1.5 mm. Tissue was subjected to 5 passes of the microneedle. Following treatment, 1.2 cm diameter skin biopsies were created and cultured at the air-liquid interface. Skin explants not subjected to microneedling served as the untreated control group. Control and microneedle control samples were cultured in Dulbecco's Modified Eagle's Medium (650 μL per well, DMEM with 10% fetal bovine serum and 1% penicillin-streptomycin) at 37° C. and 5% CO2. Treated microneedle samples with the peptides were cultured in DMEM with 10% fetal bovine serum, 1% penicillin-streptomycin. 500 uM of each peptide was dissolved into the media. Treatment medium was changed every other day with the exception of weekends. Following a 7-day culture period, all biopsies were processed for histological and immunohistochemical analysis. Skin explants were processed for hematoxylin and eosin (H&E) staining and immunohistochemical staining against filaggrin (FLG) and transglutaminase 1 (TGM1) according to standard protocol (HistoWiz, Brooklyn, NY).

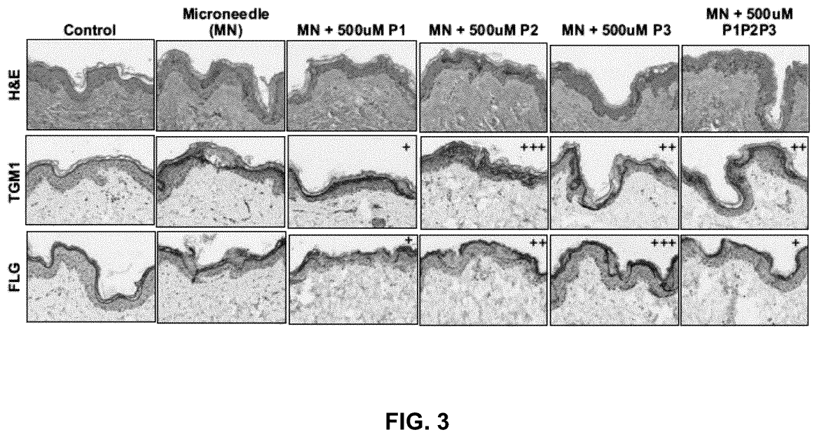

Histological analysis reveals the overall tissue structure and health. Samples subjected to microneedle treatment demonstrate presence of micropunctures, as illustrated in FIG. 3. There is no evidence of vacuole formation and epidermal cellular stress across all samples.

Transglutaminase-1 is an enzyme typically expressed localized to the upper stratum granulosum layer which is involved in the formation of the cornified cell envelope. Typical localization is demonstrated by the red staining in the untreated control. Microneedling treatment reduces expression in the microwound region. Treatment with 500 μM of Peptide 1, Peptide 2, and Peptide 3 alone and in combination increase the expression of TGM1 and localization. Meanwhile, Filaggrin is a protein essential for epidermal differentiation and is critical for proper skin barrier function. It is typically expressed in the stratum corneum and upper stratum granulosum layer, as demonstrated by the red staining in the untreated control. Similarly to TGM1, microneedling disrupts the expression of filaggrin around the microwound area due to disrupted skin barrier. Treatment with 500 μM of Peptide 1, Peptide 2, and Peptide 3 alone and in combination increase filaggrin expression. Together with the TGM1 data, this suggests that the peptides can play a role in promoting barrier renewal and barrier function following microneedling.

### Example 3

### Ex Vivo Chronic Inflammation Model

Fresh post-abdominoplasty normal human skin samples were acquired from BioIVT Inc. (Westbury, NY). Tissue was defatted and cleaned of blood residue. 1.2 cm diameter skin biopsies were created and cultured at the air-liquid interface. Skin explants not subjected to the inflammatory stimulant served as the untreated control group. Control samples were cultured in Dulbecco's Modified Eagle's Medium (650 μL per well, DMEM with 10% fetal bovine serum and 1% penicillin-streptomycin) at 37° C. and 5% CO2. Treated microneedle samples with the peptides were cultured in DMEM with 10% fetal bovine serum, 1% penicillin-streptomycin with a 2× dilution of cell stimulation cocktail (CSC) 500×. Samples treated with the peptide contained 500 μM of each peptide dissolved into the media. Fresh stimulants were made each time replenishing the treatment medium every other day with the exception of weekends. Following a 7-day culture period, all biopsies were processed for histological and immunohistochemical analysis. Skin explants were processed for hematoxylin and eosin (H&E) staining and immunohistochemical staining against filaggrin (FLG) and transglutaminase 1 (TGM1) according to standard protocol (HistoWiz, Brooklyn, NY).

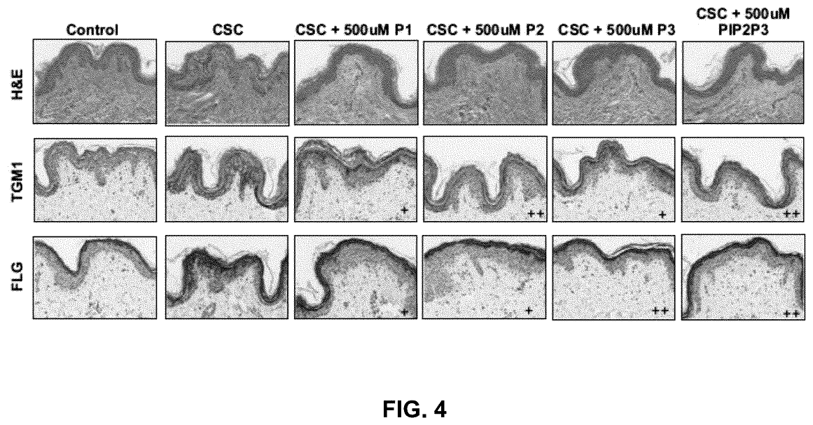

Histological analysis reveals the overall tissue structure and health, as shown in FIG. 4. There is no evidence of vacuole formation and epidermal cellular stress across all samples in the untreated control. However, treatment with the inflammatory stimulant, CSC, demonstrates signs of epidermal stress, with signs of spongiosis/vacuole formation. This effect is ameliorated with the treatment of 500 μM of Peptide 1, Peptide 2, and Peptide 3 alone and in combination.

Impaired barrier function is typically manifested in different inflammatory skin conditions. This is because there has been suggested to be a close link with the release of different pro-inflammatory cytokines with abnormal or altered lipid organization and disruption to key barrier-related proteins. Therefore, it is expected to see altered barrier markers in an inflamed skin state. In the samples treated with the CSC inflammation stimulant, there is an observed altered expression of TGM1 and filaggrin (increased expression and localization). However, treatment with the peptides alone and in combination demonstrate a phenotype similar to our untreated control, suggesting these peptides may reduce inflammation-associated barrier disruption.

### Example 4

### Face Treatment

An example face treatment containing about 0.01 to about 1 wt. % of Peptide I, Peptide II, Peptide III, or combinations thereof, is set forth below.

TABLE 6

| INGREDIENTS | Wt. % |
|---|---|
| PEPTIDE I, II, III, OR COMBINATIONS THEREOF | 0.01-10 |
| METHYLPARABEN | 0.3 |
| 2-PHENOXYETHANOL | 0.5 |
| SODIUM POLYACRYLATE | 0.8 |
| XANTHAN GUM | 0.2 |
| WATER/AQUA | 98.2 |

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both an emollient and a nonionic surfactant. If a particular composition includes both an emollient and a nonionic surfactant, a single compound will serve as only the emollient or only as the nonionic surfactant (the single compound does not simultaneously serve as both the emollient and nonionic surfactant).

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A method for treating skin in need thereof comprising topically applying a therapeutically effective amount of one or more bioactive walnut peptides or a composition comprising the one or more bioactive walnut peptides to the skin.

2. The method of claim 1, wherein the method improves skin barrier function or reepithelization of the skin.

3. The method of claim 1, wherein the method treats damaged skin.

4. The method of claim 3, wherein the damaged skin has been damaged by physical damage, chemical damage, environmental damage, sun damage, damage caused by disease, or combinations thereof.

5. The method of claim 1, wherein the method improves wound healing and comprises:
(i) damaging skin tissue using a chemical, a laser, or physical force; and
(ii) applying a therapeutically effective amount of one or more bioactive walnut peptides to the damaged skin.

6. The method of claim 5, wherein the skin tissue is damaged using an ablative laser procedure, a nonablative laser procedure, a microneedle procedure, a cryotherapy procedure, a radiofrequency microneedle procedure, dermabrasion, a chemical peel, an exfoliant, or a mechanical device that damages the skin.

7. The method of claim 1, wherein the method treats a condition, disease, or disorder of the skin selected from psoriasis, dermatitis, atopic dermatitis, allergic dermatitis, eczema, spongiosis, edema, hereditary ichthyosis, senile xerosis, palmar hyperkeratosis, plantar hyperkeratosis, cuts, bruises, pore size, skin cancer, healing or reepithelization disorders, keloids, hypertrophic scars, cellulitis, orange peel skin, elastosis, actinic elastosis, keratosis, rosacea, telangiectasia, couperosis, or combinations thereof.

8. The method of claim 1, wherein the skin in need of treatment is inflamed or suffering from an inflammatory condition, wherein optionally, the inflammatory condition is selected from atopic dermatitis, psoriasis, or a combination thereof.

9. The method of claim 1, wherein the one or more walnut peptides is combined with one or more additional skin active agents.

10. The method of claim 1, wherein the one or more bioactive walnut peptides have molecular weight of less than 5,000 Da.

11. The method of claim 10, wherein the one or more bioactive walnut peptides have 2 to 20 amino acid residues.

12. The method of claim 11, wherein the one or more bioactive walnut peptides include one or more amino acids selected from leucine, proline, and combinations thereof.

13. The method of claim 12, wherein the one or more bioactive walnut peptides include three or more amino acids selected from leucine, proline, or combinations thereof.

14. The method of claim 1, wherein the one or more bioactive walnut peptides comprise an amino acid sequence selected from Leu-Pro-Leu, Leu-Leu-Pro, Pro-Pro-Leu, or Pro-Leu-Pro.

15. The method of claim 14, wherein the one or more bioactive walnut peptides comprise an amino acid sequence selected from Tyr-Val-Leu-Leu-Pro-Ser-Pro-Lys or Lys-Val-Pro-Pro-Leu-Leu-Tyr.

16. The method of claim 1, wherein the one or more bioactive walnut peptides are selected from:
Peptide I
TWLPLPR (Thr-Trp-Leu-Pro-Leu-Pro-Arg),
Peptide II
YVLLPSPK (Tyr-Val-Leu-Leu-Pro-Ser-Pro-Lys),
Peptide III
KVPPLLY (Lys-Val-Pro-Pro-Leu-Leu-Tyr),
or combination thereof.

17. The method claim 1, wherein the method comprises topically applying a combination of two or more bioactive walnut peptides to the skin.

18. The method of claim 1, wherein the method:
(a) potentiates ceramide synthesis (ACER1);
(b) facilitates terminal differentiation (involucrin (IVL), Loricrin (Lor), and Transglutaminase 1 (TGM1));
(c) increases keratinization (KRT);
(d) promotes late cornified cell envelope formation (LCE); and/or
(e) increases abundance or synthesis of small proline rich proteins (SPRR).

19. The method of claim 1, wherein the method comprises topically applying a combination of two or more bioactive walnut peptides to the skin, wherein topical application of the combination of the two or more walnut peptides provides a synergistic effect that is greater than the additive sum provided by topical application of corresponding amounts of the individual bioactive walnut peptides.

20. A pharmaceutical or cosmetic composition comprising:
(a) a therapeutically effective amount of one or more bioactive walnut peptides; and
(b) a physiologically acceptable.
